(19) United States
(12) Patent Application Publication
(10) Pub. No.: US 2006/0100715 A1
(43) Pub. Date: May 11, 2006

(54) Arthroplasty implant

(76) Inventor: Malan De Villiers, Irene (ZA)

Correspondence Address: PAULEY PETERSEN & ERICKSON, 2800 WEST HIGGINS ROAD, SUITE 365, HOFFMAN ESTATES, IL, 60195, US

(21) Appl. No.: 10/527,656

(22) PCT Filed: Sep. 19, 2003
(86) PCT No.: PCT/IB03/04051

§ 371 Date: Dec. 30, 2005

(30) Foreign Application Priority Data
Sep. 19, 2002 (ZA) 2002/7516

(51) Int. Cl. A61F 2/30; A61F 2/42

(52) U.S. Cl. 623/23.4; 623/21.11

(57) ABSTRACT

The invention concerns an arthroplasty implant for providing a joint between first and second members of the body, such as a metatarsophalangeal (MTP) joint, a metacarpophalangeal (MCP) joint or a proximal interphalangeal (PIP) joint. The implant of the invention includes a fist component (10) defining a concave surface (14), a second component (20) defining a convex surface (24) and an intermediate component (32) which is located between the first and second components. The intermediate component has a convex surface (36) which is slidable on the concave surface of the first component to allow articulation between the first component and the intermediate component and a concave surface slidable on the convex surface of the second component to allow articulation between the second component and the intermediate component. A means (16, 38; 50, 60) is provided to prevent the intermediate component from separating laterally from at least one of the first and second components.

### BACKGROUND TO THE INVENTION

THIS invention relates to an arthroplasty implant.

The invention is particularly concerned with arthroplasty implants of the wrist and small bones of the hand and foot, such as metatarsophalangeal (MTP) joint implants, metacarpophalangeal (MCP) joint implants and proximal interphalangeal (PIP) joint implants.

Various types of implants for such joints have been proposed and are in use. It is however believed that the known implants, most of which are of two part construction, suffer from one disadvantage or other that either limits their flexibility, load-transmitting ability or life expectancy.

### SUMMARY OF THE INVENTION

According to the present invention there is provided an arthroplasty implant for providing a joint between first and second members of the body, the implant comprising:
a first component defining a concave surface and having first connection means for connecting it to the first body member;
a second component defining a convex surface and having second connection means for connecting it to the second body member;
an intermediate component for location between the first and second components and defining a convex surface which is slidable on the concave surface of the first component to allow articulation between the first component and the intermediate component and a concave surface slidable on the convex surface of the second component to allow articulation between the second component and the intermediate component, and
means for preventing the intermediate component from separating laterally from at least one of the first and second components.

The concave surface of the first component and the convex surface of the intermediate component are preferably complementally, spherically curved. In the preferred embodiments, the convex surface of the second component and the concave surface of the intermediate component are defined by radii of curvature which differ in mutually orthogonal directions. The length of the convex surface in a direction defined by a relatively large radius of curvature is preferably greater than the length of that surface in a direction defined by a relatively small radius of curvature.

In all cases, the first and second components should be capable of translation and articulation relative to the intermediate component.

One embodiment of the invention comprises a central projection on the concave surface of the first component and a central opening in the convex surface of the intermediate component, the projection in use locating loosely in the opening to prevent lateral separation of the intermediate and first components.

In another embodiment of the invention one of the first component and the intermediate component includes a laterally outwardly facing projection and the other of the first component and the intermediate component includes a laterally inwardly facing recess, the projection in use interacting with the recess to prevent lateral separation of the intermediate and first components. Typically in this embodiment, the first component includes an annular wall bounding the concave surface of that component, the peripheral wall being formed with an annular undercut defining the laterally inwardly facing recess, and the intermediate component includes an annular rib defining the laterally outwardly facing projection, interaction in use between the rib and the undercut preventing lateral separation of the intermediate and first components.

Other features of the invention are defined in the appended claims.

### BRIEF DESCRIPTION OF THE DRAWINGS

The invention will now be described in more detail, by way of example only, with reference to the accompanying drawings in which:

FIG. 1 shows a perspective view of a first component of an arthroplasty implant according to a first embodiment of the invention;

FIG. 2 shows a side view of the component seen in FIG. 1;

FIG. 3 shows a perspective view of a second component of an arthroplasty implant according to first embodiment of the invention;

FIG. 4 shows a side view of the component seen in FIG. 3;

FIG. 5 shows a plan view of the component seen in FIG. 3;

FIG. 6 shows a perspective view of an intermediate component of an arthroplasty implant according to the first embodiment of the invention;

FIG. 7 shows a plan view of the intermediate component seen in FIG. 6;

FIG. 8 shows a cross-section at the line 8-8 in FIG. 7;

FIG. 9 shows a cross-section at the line 9-9 in FIG. 7;

FIG. 10 shows a side view of an assembled arthroplasty implant according to the first embodiment of the invention with the first and intermediate components in a neutral position before articulation between them;

FIG. 11 shows a similar side view of the assembled arthroplasty implant seen in FIG. 10 after maximum articulation between the first and intermediate components;

FIG. 12 shows a plan view of the assembled arthroplasty implant seen in FIG. 10 after maximum articulation between the first and intermediate components.

FIG. 13 shows a perspective view of the first component of an arthroplasty implant according to a second embodiment of the invention;

FIG. 14 shows a side view of the first component seen in FIG. 13;

FIG. 15 shows a perspective view of the intermediate component of an arthroplasty implant according to the second embodiment of the invention;

FIG. 16 shows a side view in the direction of the arrow 16 of the intermediate component seen in FIG. 15;

FIG. 17 shows a side view in the direction of the arrow 17 of the intermediate component seen in FIG. 15;

FIG. 18 shows a side view of an assembled arthroplasty implant according to the second embodiment of the invention with the first and intermediate components in a neutral position before articulation between them; and

FIG. 19 shows a similar side view of the assembled arthroplasty implant seen in FIG. 18 after maximum articulation between the first and intermediate components.

### DESCRIPTION OF PREFERRED EMBODIMENTS

The drawings illustrate individual components of preferred metatarsophalangeal (MTP) joint implants, and the assembled MTP implants. In each case the implant consists of three individual components.

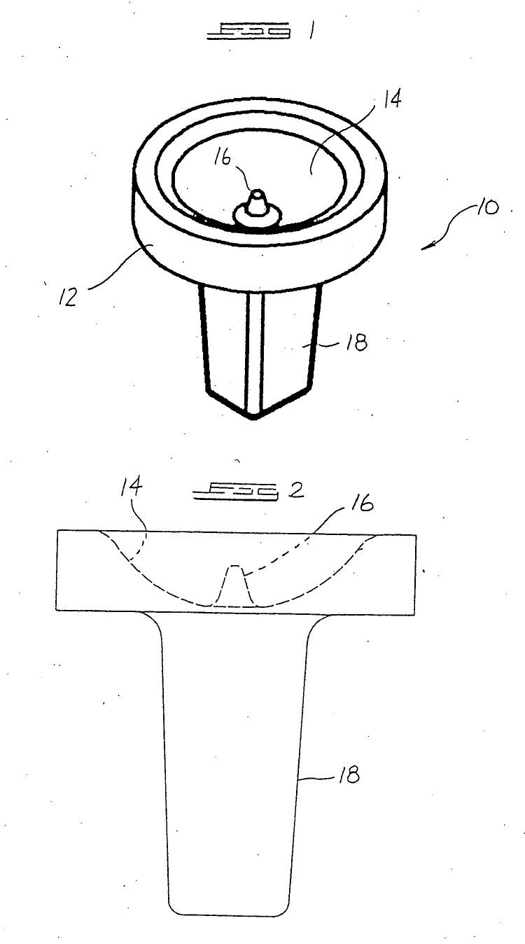

A first embodiment of the invention is illustrated in FIGS. 1 to 12 of the drawings. In this embodiment, FIGS. 1 and 2 illustrate a first, phalangeal component 10 which is connected in use to a phalanx. It includes a body 12 formed with a spherically curved, concave surface 14. Projecting centrally from the surface 14 is a conical peg 16 and projecting rearwardly from the body 12 is a tapered post 18 of square cross-section. In use, the post 18 is placed and anchored in a predrilled hole in the phalanx.

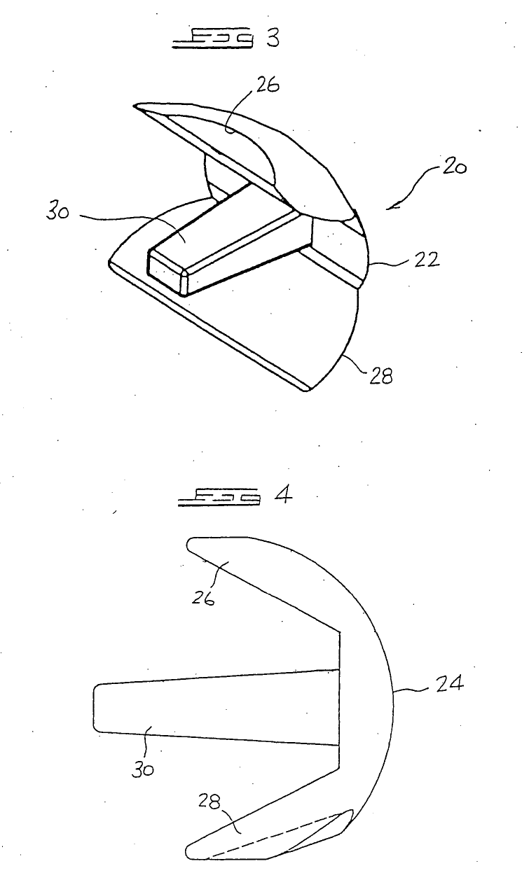

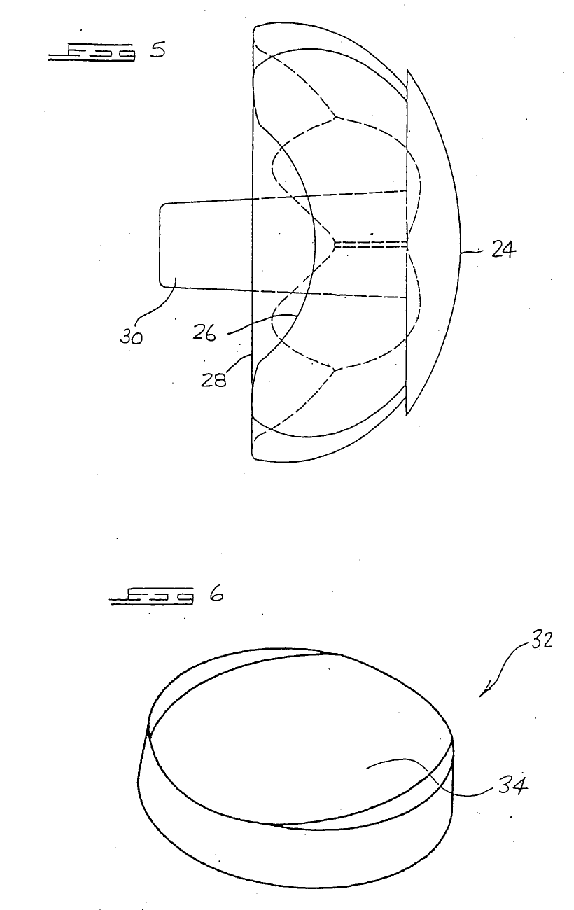

FIGS. 3 to 5 illustrate a second, tarsal component 20 which is connected in use to the associated tarsus. It includes a body 22 with a convexly curved surface 24 and curved skirts 26, 28. The radius of curvature of the surface 24 in the view of FIG. 4 is less than the radius of curvature in the view of FIG. 5. Projecting rearwardly from the body 22 is a tapered post 30 of square cross-section. In use the post 30 is placed and anchored in a predrilled hole in the tarsus.

Both the phalangeal component 10 and the tarsal component 20 are made in one piece of grade 5 titanium, their curved surfaces 14 and 24 being provided with a titanium nitride finish.

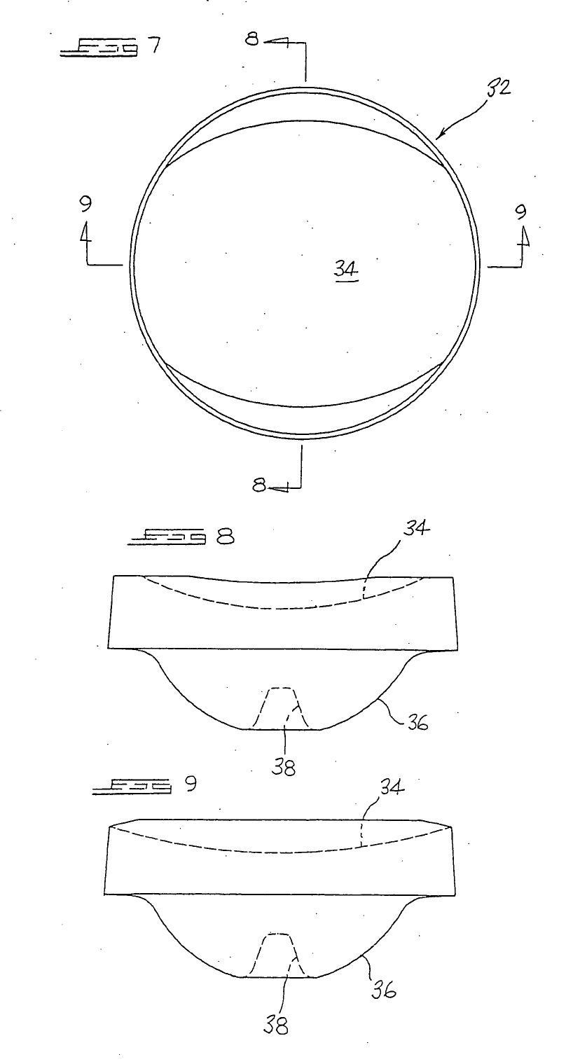

FIGS. 6 to 9 illustrate an intermediate component in the form of a meniscus 32 which is located in the assembled MTP implant between the phalangeal and tarsal components 10 and 20. The meniscus 32 is made of a low friction plastic material, in this case an ultra high molecular weight polyethylene (UHMWPE) available under the name ORTHOSOL™. One side of the meniscus is formed with a concave surface 34 and the opposite side with a convex surface 36. The convex surface is spherically curved and is formed centrally with a conical recess or socket 38. The concave surface 34 is not spherical. The radius of curvature of the surface 34 in FIG. 8, which matches radius of curvature of the surface 24 in FIG. 4, is less than the radius of curvature of the surface 34 in FIG. 9, which matches the radius of curvature of the surface 24 in FIG. 5. It will accordingly be understood that the concave surface 34 of the meniscus is complemental to the convex surface 24 of the tarsal component 20, and that the convex surface 36 of the meniscus is complemental to the concave surface 14 of the phalangeal component 10.

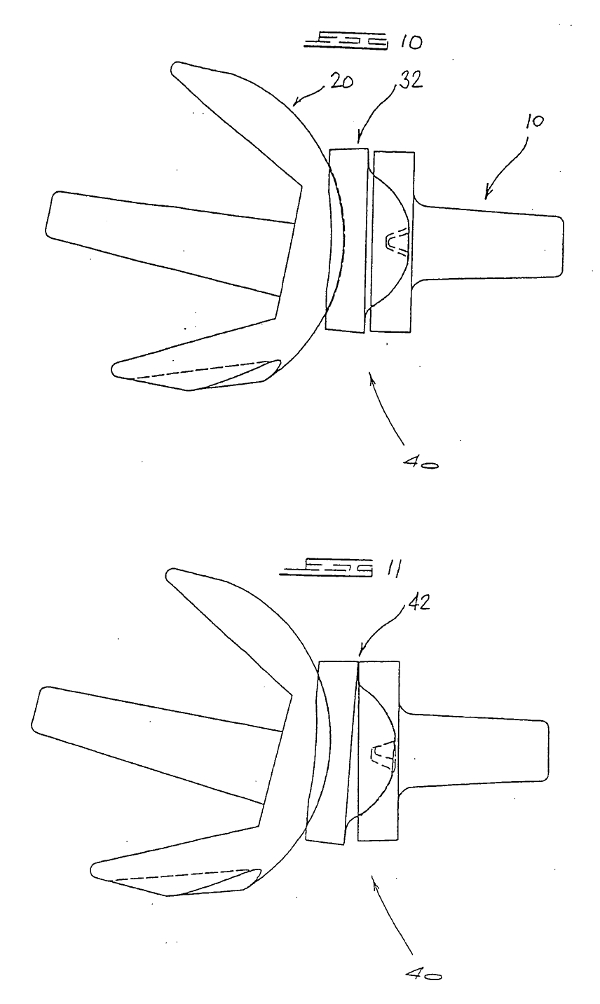

FIGS. 10 to 12 illustrate an assembled MTP arthroplasty implant 40, according to the first embodiment of the invention, and consisting of the three components 10, 20 and 32. The meniscus 32 is located between the phalangeal and tarsal components 10 and 20 with the various concave and convex surfaces in cooperating relationship with one another. The peg 16 of the phalangeal component is located in the socket 38 of the meniscus 32. In this regard it will be noted that the transverse dimension of the peg is somewhat less than the transverse dimension of the socket at any given point along the length of the peg and socket.

In FIG. 10, the phalangeal component 10 and the meniscus 32 are at a neutral orientation with one another, i.e. they are axially aligned and no articulation or translation has taken place between them. FIG. 11 illustrates the situation after maximum permitted articulation and translation has taken place between these components. It will be noted that in FIG. 11, edge regions of the phalangeal component 10 and meniscus 32 come into contact with one another, as indicated by the arrow 42. Further articulation in the same sense past this condition is impossible. The fact that the socket 38 is oversize with respect to the peg 16 permits translation and maximum articulation to take place, but it will be noted that in FIG. 11 the peg 16 also abuts the side of the socket 38 to prevent further articulation or translation.

Throughout the permitted range of movement between the phalangeal component and the meniscus, the peg 16 remains located in the socket 38. This prevents the meniscus from separating laterally from the phalangeal component, i.e. holds the meniscus captive relative to the phalangeal component at all times.

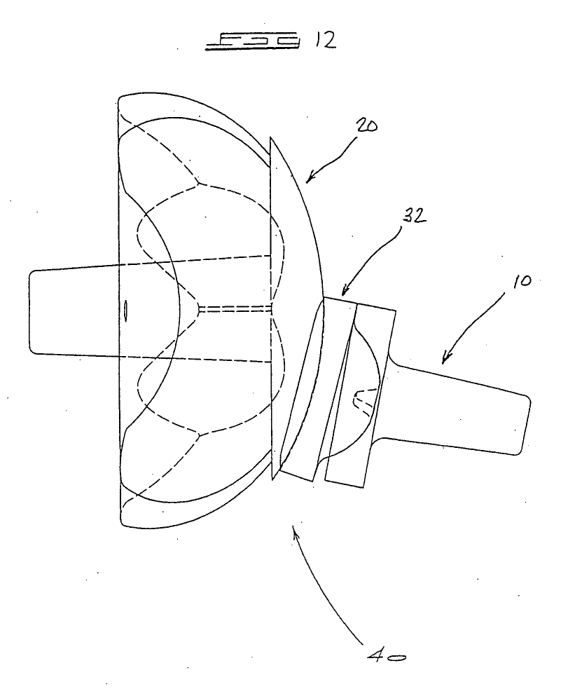

In FIGS. 10 and 11, there is no change in the positional relationship of the meniscus and the tarsal component 20. Given the complemental curvature of their respective convex and concave surfaces, it will however be understood that these components are free to slide over one another and, in doing so, to articulate relative to one another. This is illustrated by FIG. 12, which shows the meniscus, and with it the phalangeal component, after relative sliding, i.e. translation, and articulation has taken place. The convex surface 24 of the tarsal component 20 is substantially larger than the complemental concave surface 34 of the meniscus, allowing translation and articulation to take place over a wide range of positions and angles.

A second embodiment of the invention is illustrated in FIGS. 13 to 19 of the drawings. In these Figures features corresponding to features seen in FIGS. 1 to 12 are indicated by the same reference numerals.

Notable differences between the first or phalangeal component 10 illustrated in FIGS. 13 and 14 and the first component 10 seen in FIGS. 1 and 2 are the absence of the central peg 16 and the inclusion of an annular, peripheral wall 52 which bounds the concave surface 14 and which is formed with an undercut 50 defining a laterally inwardly facing recess. The wall 52 and concave surface in combination define a cup-shaped receptacle 53.

Another difference between the component 10 of FIGS. 13 and 14 and that of FIGS. 1 and 2 is the face that the post 18 has a round cross-section and is provided at its end with barb formations 54.

The second or tarsal component 20 of the second embodiment is seen in FIGS. 18 and 19. This component differs from the second component illustrated in FIGS. 3 to 5 in that the post 30 is of round cross-section and carries barb formations 56 at its end. The structure defining the convex surface 24 is also slightly different, as illustrated.

It is believed that the barb formations 54 and 56 will be able to provide better anchorage of the posts 18 and 30 in their respective predrilled holes in the phalanx and tarsus respectively.

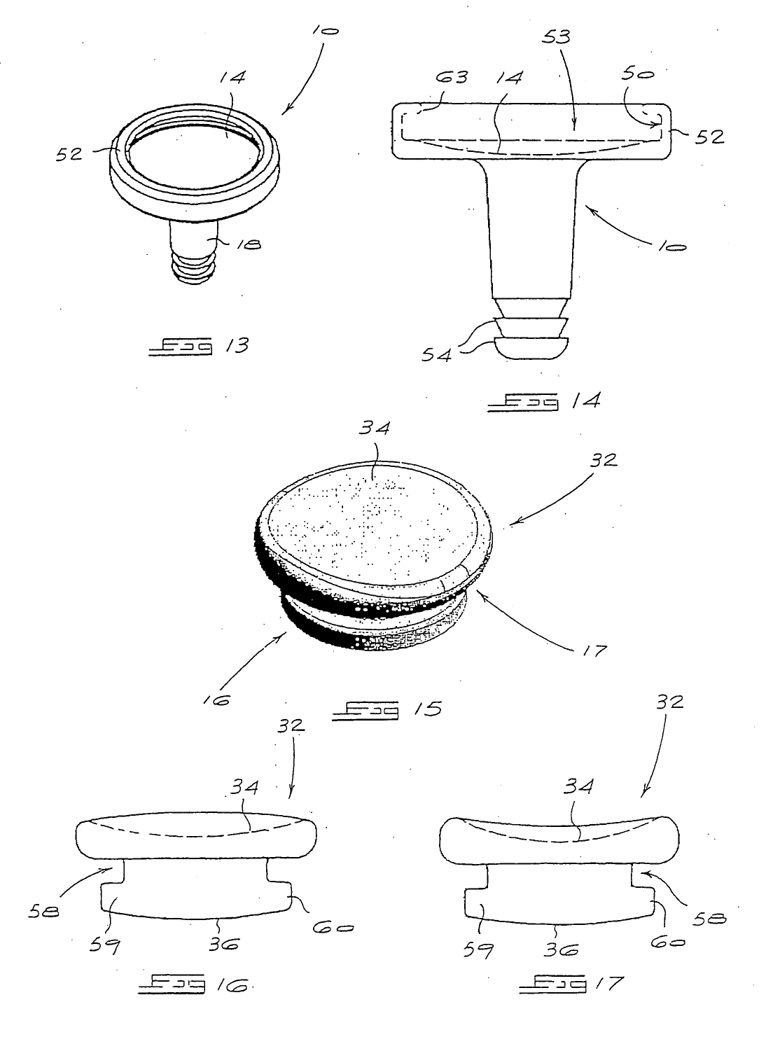

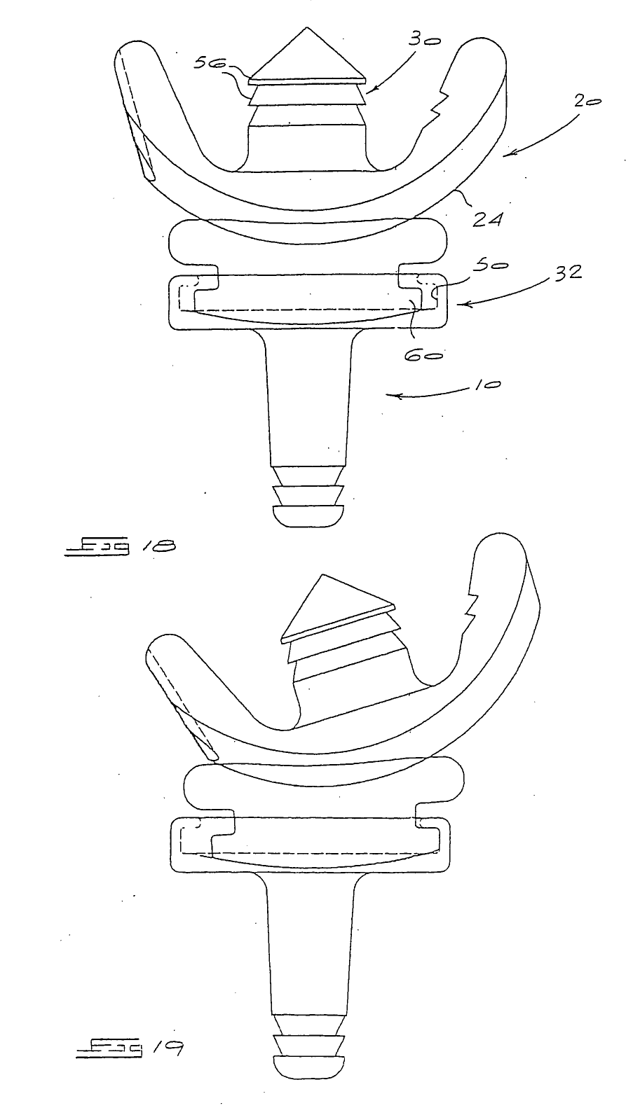

The intermediate component or meniscus 32 of the second embodiment is illustrated in FIGS. 15 to 17. As will be apparent from FIGS. 16 and 17, the concave surface 34 of this meniscus is similar to that of the first embodiment. The convex surface 36 of the second embodiment is however defined by a somewhat greater radius of curvature than the corresponding surface in the first embodiment. Anther difference between the convex surface 36 of the second embodiment and that of the first embodiment is the absence of the central recess or socket 38. The meniscus also cinludes an annular groove 58, with a portion 59 of the meniscus beneath this groove presenting an annular, outwardly facing rib 60.

FIGS. 18 and 19 illustrated the second embodiment in an assembled condition. The portion 59 of the meniscus is received in the cup-shaped receptacle 53. Typically the outer diameter of the rib 60 will be such that the portion 59 is either be a very close fit or a press fit through the opening defined by the inner rim 63 of the undercut side wall 52. A comparison of FIGS. 18 and 19 shows how the first component and meniscus can both articulate and translate laterally relative to one another. It will however be understood that throughout the range of permitted translation and articulation, the lower portion 59 of the meniscus is held captive relative to the first component 10 by the undercut side wall 52. Thus in this embodiment the interaction of the rib 60 and the undercut recess 50 prevents the components from separating laterally from one another.

A comparison of FIGS. 18 and 19 also illustrates the ability of the implant to accommodate a wide range of translation and articulation between the second component 20 and the meniscus.

Referring again to the first embodiment, a comparison of FIGS. 10 and 11 on one hand and FIG. 12 on the other hand indicates that articulation and translation between the components can take place in mutually orthogonal directions. The range of translation and articulated movement between the tarsal component 20 and the meniscus in one direction, illustrated by FIGS. 10 and 11, is greater than the corresponding range of movement in the orthogonal direction, illustrated by FIG. 12. This feature is attributable to the shape of the structure defining the convex surface 24 and is provided for the reason that most small joints of the hand or foot are designed to flex primarily in one direction. Considering, for instance, a toe joint, the primary flexural movement of the toe is towards or away from the foot rather than at right angles thereto, although at least a small degree of movement in the latter sense must also be accommodated.

FIGS. 18 and 19 only illustrate the primary flexural movement, but it will be understood that a similar range of movement in the orthogonal direction is also possible in this embodiment.

In both embodiments the concave surface 34 of the meniscus and the convex surface of the tarsal component 20 is defined by radii of curvature which differ from one another in mutually orthogonal direction. This feature also contributes to movement in the primary direction. In the case of the first embodiment compare, for instance, FIGS. 4 and 5 illustrating the tarsal component and FIGS. 8 and 9 illustrating the meniscus. In the case of the second component, a similar comparison may be made between FIGS. 16 and 17 illustrating the meniscus. In each case, it will be understood that sliding movement is easier in the direction defined by the larger radius of curvature, i.e. the more gentle curvature.

It is believed that the three component implants described above and illustrated in the drawings will provide for substantial flexibility in the implanted arthroplasty. Also, the relatively large bearing areas between the respective components will, it is believed, provide the arthroplasty with substantial longevity. Referring in particular to the phalangeal components 10 and the menisci 32, the fact that these components are retained in their cooperating relationship either by the interaction of the peg 16 and socket 38 or by the interaction of the portion 60 and the undercut 50 means that there still remains a large bearing area between the components to transmit generally axial loading.

Many modifications are possible within the scope of the invention. For instance, although it is considered beneficial in the first embodiment for the peg to abut the side of the socket, as illustrated in FIG. 11, this is not critical to the performance of the implant. In the second embodiment described above, it is the interaction of an outwardly facing projection, i.e. the rib 60 and the inwardly facing recess, i.e, the undercut 50, which prevents lateral separation of the phalangeal component and meniscus. The rib and recess are included in the phalangeal component and meniscus respectively. It will however be understood that the situation could equally well be reversed, with a projection on the phalangeal component interacting with a recess on the intermediate member. In similar fashion the peg and recess arrangement of the first embodiment could be reversed with the peg on the intermediate component and the recess in the first or phalangeal component. Still further the concave and convex surfaces could be reversed so that, for instance, the palangeal component has the convex surface and the tarsal component has the concave surface, such surfaces interacting with a concave and convex surfaces respectively on the intermediate member. It is also feasible for means to be provided, eg in the form of the described peg and recess combination or the described rib and recess combination, to prevent separation of the intermediate member from both of the first and second components. It is the intention that all such variations are included with the scope of the invention.

Further, while specific mention has been made of MTP arthroplasty implants, it will be understood that the principles of the invention are equally applicable to other arthroplasty implants, including those mentioned at the outset, typically for the wrist or small bones of the hand or foot.

1-14. (canceled)

15. An arthroplasty implant for providing a joint between a first body member and a second body member, the arthroplasty implant comprising:
a first component defining a concave surface and having a first connector connecting the first component to the first body member;
a second component defining a convex surface and having a second connector connecting the second component to the second body member; and
an intermediate component positioned between the first component and the second component and defining a convex surface slidable on the concave surface of the first component to allow articulation between the first component and the intermediate component and a concave surface slidable on the convex surface of the second component to allow articulation between the second component and the intermediate component.

16. The arthroplasty implant according to claim 15 wherein the concave surface of the first component and the convex surface of the intermediate component are complementally, spherically curved.

17. The arthroplasty implant according to claim 15 wherein each of the convex surface of the second component and the concave surface of the intermediate component is defined by radii of curvature which differ in mutually orthogonal directions.

18. The arthroplasty implant according to claim 15 wherein a length of the convex surface of the second component in a direction defined by a relatively large radius of curvature is greater than a length of the convex surface in a direction defined by a relatively small radius of curvature.

19. The arthroplasty implant according to claim 15 wherein each of the first component and the second component is capable of translation and articulation relative to the intermediate component.

20. The arthroplasty implant according to claim 15 wherein each of the first component and the second component is made of grade 5 titanium and each of the concave surface and the convex surface has a titanium nitride finish.

21. The arthroplasty implant according to claim 15 wherein the intermediate component is made of a low friction plastic material.

22. The arthroplasty implant according to claim 21 wherein the plastic material is ultra high molecular weight polyethylene.

23. The arthroplasty implant according to claim 15 wherein the first connector and the second connector each includes a projecting post locatable in a hole formed in a respective body member.

24. The arthroplasty implant according to claim 15 further comprising a central projection on the concave surface of the first component and a central opening in the convex surface of the intermediate component, the central projection positionable within the central opening to prevent lateral separation of the intermediate component and the first component.

25. The arthroplasty implant according to claim 24 wherein the concave surface of the first component is bounded by a first peripheral edge and the convex surface of the intermediate component is bounded by a second peripheral edge, the first peripheral edge contacting the second peripheral edge when relative movement between the first component and the intermediate component reaches a maximum limit.

26. The arthroplasty implant according to claim 15 wherein one of the first component and the intermediate component includes a laterally outwardly facing projection and the other of the first component and the intermediate component includes a laterally inwardly facing recess, the projection interacting with the recess to prevent lateral separation of the intermediate component and the first component.

27. The arthroplasty implant according to claim 15 wherein the first component includes an annular wall bounding the concave surface of the first component, the annular wall being formed with an annular undercut defining a laterally inwardly facing recess, and the intermediate component includes an annular rib defining a laterally outwardly facing projection, interaction between the annular rib and the annular undercut preventing lateral separation of the intermediate component and the first component.

28. The arthroplasty implant according to claim 15 wherein the first component is a phalangeal component of a metatarsophalangeal joint implant and is connectable to a phalanx, and the second component is a tarsal component of the metatarsophalangeal joint implant and is connectable to a tarsus.

29. The arthroplasty implant according to claim 15 wherein the second component is movable with respect to the intermediate component in mutually orthogonal directions.

30. An arthroplasty implant for providing a joint between a first body member and a second body member, the arthroplasty implant comprising:
a first component defining a concave surface and having a first connector connecting the first component to the first body member;
a second component defining a convex surface and having a second connector connecting the second component to the second body member;
an intermediate component positioned between the first component and the second component and defining a convex surface slidable on the concave surface of the first component to allow articulation between the first component and the intermediate component and a concave surface slidable on the convex surface of the second component to allow articulation between the second component and the intermediate component; and
a central projection formed on the concave surface of the first component and a central opening formed in the convex surface of the intermediate component, the central projection positionable within the central opening and preventing lateral separation of the intermediate component and the first component.

31. The arthroplasty implant according to claim 30 wherein the concave surface of the first component is bounded by a first peripheral edge and the convex surface of the intermediate component is bounded by a second peripheral edge, the first peripheral edge contacting the second peripheral edge as relative movement between the first component and the intermediate component reaches a maximum limit.

32. An arthroplasty implant for providing a joint between a first body member and a second body member, the arthroplasty implant comprising:
a first component defining a concave surface and having a first connector connecting the first component to the first body member;
a second component defining a convex surface and having a second connector connecting the second component to the second body member;
an intermediate component positioned between the first component and the second component and defining a convex surface slidable on the concave surface of the first component to allow articulation between the first component and the intermediate component and a concave surface slidable on the convex surface of the second component to allow articulation between the second component and the intermediate component, one of the first component and the intermediate component including a laterally outwardly facing projection and the other of the first component and the intermediate component including a laterally inwardly facing recess, the projection interacting with the recess and preventing lateral separation of the intermediate component and the first component.

33. The arthroplasty implant according to claim 32 wherein the first component includes an annular wall bounding the concave surface of the first component, the annular wall being formed with an annular undercut defining the laterally inwardly facing recess, and the intermediate component includes an annular rib defining the laterally outwardly facing projection, interaction between the annular rib and the annular undercut preventing lateral separation of the intermediate component and the first component.

34. The arthroplasty implant according to claim 32 wherein the first connector and the second connector each includes a central projecting post locatable in a hole formed in a respective body member.
